(19) United States
(12) Patent Application Publication
(10) Pub. No.: US 2026/0182978 A1
(43) Pub. Date: Jul. 2, 2026

(54) IMPLANT FOR OCCLUDING A LEFT ATRIAL APPENDAGE

(71) Applicant: BOSTON SCIENTIFIC SCIMED, INC., MAPLE GROVE, MN (US)

(72) Inventors: Philip Andrew Litecky, Forest Lake, MN (US); Troy Anthony Giese, Blaine, MN (US); Patrick Willoughby, Shoreview, MN (US)

(73) Assignee: BOSTON SCIENTIFIC SCIMED, INC., MAPLE GROVE, MN (US)

(21) Appl. No.: 19/376,042

(22) Filed: Oct. 31, 2025

Related U.S. Application Data
(60) Provisional application No. 63/714,515, filed on Oct. 31, 2024.

(51) Int. Cl. A61B 17/00

(52) U.S. Cl. CPC A61B 17/0057; A61B 2017/00588; A61B 2017/00632; A61B 2017/00646

(57) ABSTRACT

An implant for occluding a left atrial appendage includes an expandable framework including a central spine and a plurality of branch layers extending outward from the spine. Each branch layer includes at least one layer of foam fixedly attached thereto. Branches within each branch layer are circumferentially misaligned with branches within other branch layers. The plurality of branch layers includes first, second, and third branch layers fixedly attached to first, second, and third ring members coupled to the spine.

### CROSS-REFERENCE TO RELATED APPLICATIONS

This application claims the benefit of priority of U.S. Provisional Application No. 63/714,515 filed Oct. 31, 2024, the entire disclosure of which is hereby incorporated by reference.

### TECHNICAL FIELD

The disclosure relates generally to medical devices and more particularly to devices, systems, and methods that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices, systems, and methods, each has certain advantages and disadvantages. There is an ongoing need to provide alternative medical devices and systems, and methods for manufacturing and using medical devices and systems.

### SUMMARY

In one example, an implant for occluding a left atrial appendage may comprise an expandable framework configured to shift from a collapsed configuration to an expanded configuration, wherein the expandable framework comprises a central spine and a plurality of branch layers extending radially outward from the central spine. Each branch layer of the plurality of branch layers may comprise at least one layer of foam fixedly attached thereto.

In addition, or alternatively, to any example described herein, each branch layer of the plurality of branch layers comprises a plurality of branches extending radially outward from the central spine.

In addition, or alternatively, to any example described herein, each branch of the plurality of branches within each branch layer extends radially outward in a different direction from the central spine.

In addition, or alternatively, to any example described herein, the at least one layer of foam is fixedly attached to a top surface of its respective branch layer.

In addition, or alternatively, to any example described herein, the at least one layer of foam is fixedly attached to a bottom surface of its respective branch layer.

In addition, or alternatively, to any example described herein, each branch layer is embedded within its respective at least one layer of foam.

In addition, or alternatively, to any example described herein, each layer of foam of the at least one layer of foam comprises a plurality of individual pieces of foam disposed circumferentially around the central spine.

In addition, or alternatively, to any example described herein, the plurality of individual pieces of foam within each layer of foam engage with each other when the expandable framework is in the expanded configuration to form a single complete layer of foam surrounding the central spine.

In addition, or alternatively, to any example described herein, each layer of foam of the at least one layer of foam comprises a single piece of foam surrounding the central spine.

In addition, or alternatively, to any example described herein, branches within each branch layer of the plurality of branch layers are circumferentially offset from branches of an adjacent branch layer of the plurality of branch layers.

In addition, or alternatively, to any example described herein, and in another example, an implant for occluding a left atrial appendage may comprise an expandable framework configured to shift from a collapsed configuration to an expanded configuration, wherein the expandable framework comprises a central spine and a plurality of branch layers extending radially outward from the central spine. Each branch layer of the plurality of branch layers may comprise at least one layer of foam fixedly attached thereto. Branches within each branch layer may be circumferentially misaligned with branches within every other branch layer.

In addition, or alternatively, to any example described herein, the plurality of branch layers is axially spaced apart from each other in the expanded configuration.

In addition, or alternatively, to any example described herein, at least one branch layer of the plurality of branch layers has an outermost radial extent in the expanded configuration that is different from any other branch layers of the plurality of branch layers.

In addition, or alternatively, to any example described herein, each branch layer comprises a plurality of branches, and each branch of the plurality of branches comprises a main limb extending radially outward from the central spine and a plurality of side branches extending laterally from the main limb.

In addition, or alternatively, to any example described herein, and in another example, an implant for occluding a left atrial appendage may comprise an expandable framework configured to shift from a collapsed configuration to an expanded configuration, wherein the expandable framework comprises a central spine and a plurality of branch layers extending radially outward from the central spine. The plurality of branch layers may comprise a first branch layer, a second branch layer axially spaced apart from the first branch layer in the expanded configuration, and third branch layer axially spaced apart from the first branch layer and the second branch layer in the expanded configuration. The first branch layer may comprise at least one layer of foam fixedly attached thereto, the second branch layer comprises at least one layer of foam fixedly attached thereto, and the third branch layer comprises at least one layer of foam fixedly attached thereto. The first branch layer may comprise a first plurality of branches fixedly attached to a first ring member coupled to the central spine, the second branch layer may comprise a second plurality of branches fixedly attached to a second ring member coupled to the central spine, and the third branch layer may comprise a third plurality of branches fixedly attached to a third ring member coupled to the central spine.

In addition, or alternatively, to any example described herein, each branch of the first plurality of branches includes a base end fixedly attached to the first ring member and a free end disposed opposite the base end.

In addition, or alternatively, to any example described herein, each branch of the first plurality of branches extends in a distal direction from the base end to the free end.

In addition, or alternatively, to any example described herein, in the collapsed configuration the free end is disposed at a first radial position relative to a central longitudinal axis of the central spine, and wherein in the expanded configuration the free end is disposed at a second radial position relative to the central longitudinal axis of the central spine that is radially outward of the first radial position.

In addition, or alternatively, to any example described herein, the first ring member is axially spaced apart from the second ring member and the third ring member.

In addition, or alternatively, to any example described herein, the expandable framework is self-biased toward the expanded configuration.

In addition, or alternatively, to any example described herein, the expandable framework is manually actuatable between the collapsed configuration and the expanded configuration.

In addition, or alternatively, to any example described herein, the expandable framework comprises a tubular member disposed radially outward of the central spine.

In addition, or alternatively, to any example described herein, longitudinal slots formed in the tubular member surround each branch of the first plurality of branches, each branch of the second plurality of branches, and each branch of the third plurality of branches.

In addition, or alternatively, to any example described herein, the central spine is axially movable relative to the tubular member to shift the expandable framework between the collapsed configuration and the expanded configuration.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-3 illustrate selected aspects of an expandable framework of an implant for occluding a left atrial appendage;

FIGS. 4-7 illustrate selected aspects of the implant of FIGS. 1-3; and

FIGS. 8-15 schematically illustrate selected aspects related to deploying the implant of FIGS. 1-7.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the disclosure. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently - such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The devices, systems, and/or methods disclosed herein may provide a number of desirable features and benefits as described in more detail below.

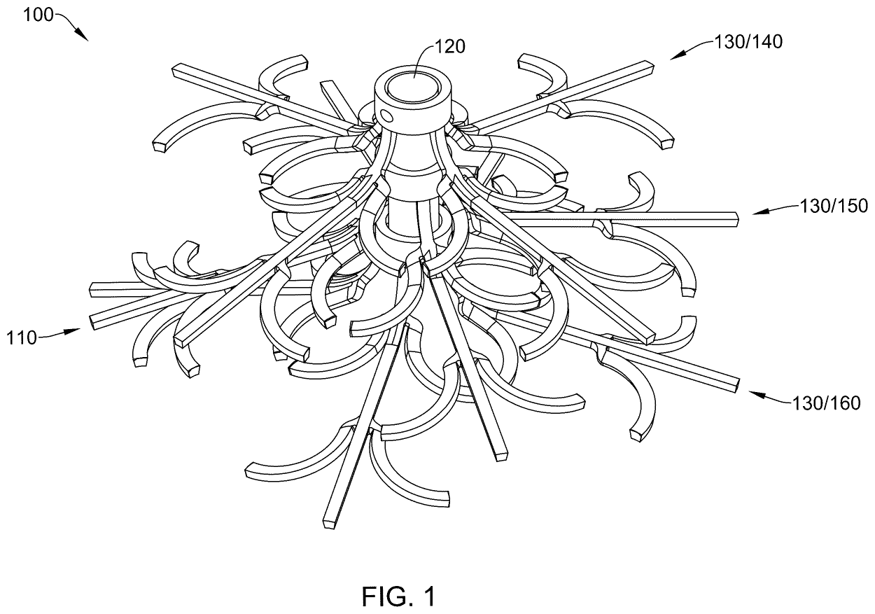

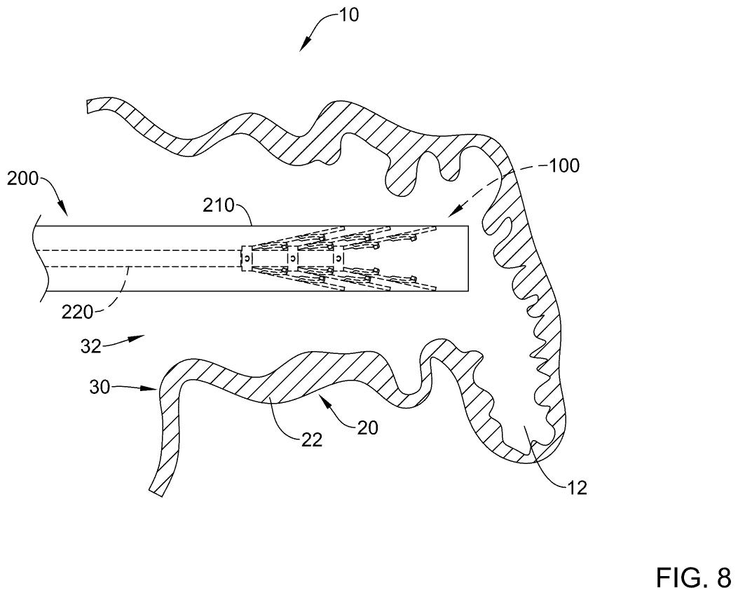

FIGS. 1-7 illustrate selected aspects of an implant 100 for occluding a left atrial appendage (e.g., FIG. 8). In some embodiments, the implant 100 may comprise an expandable framework 110 configured to shift from a collapsed configuration (e.g., FIG. 8) to an expanded configuration (e.g., FIGS. 1, 10, 12). In some embodiments, the expandable framework 110 may comprise a central spine 120 and a plurality of branch layers 130 extending radially outward from the central spine 120.

In some embodiments, the plurality of branch layers 130 may comprise a first branch layer 140, a second branch layer 150, and a third branch layer 160. In some embodiments, the implant 100 may be modular and/or adjustable to accommodate different sizes, lengths, and/or depths of left atrial appendages. For example, in some embodiments, the plurality of branch layers 130 may comprise two branch layers, three branch layers, four branch layers, five branch layers, etc. as desired and/or required to occlude and/or fill the left atrial appendage. Other configurations are also contemplated.

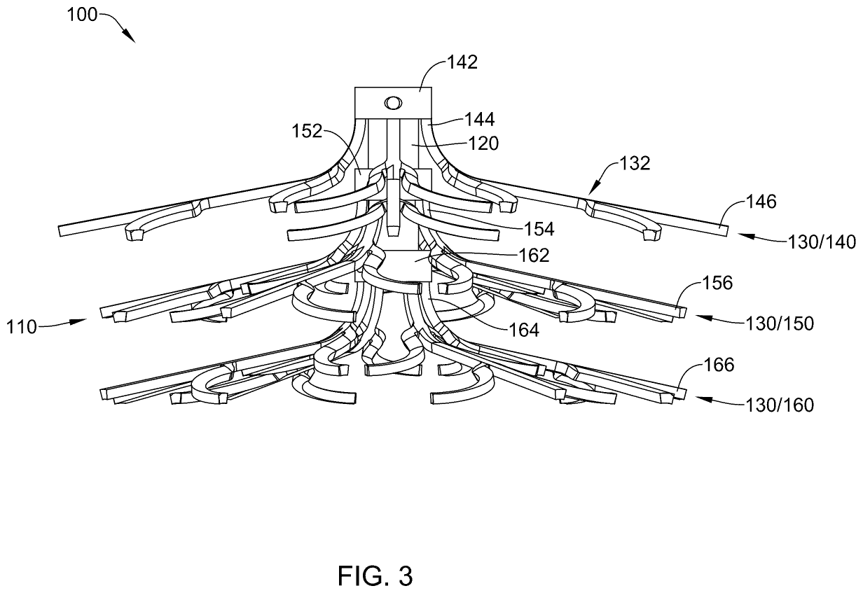

In some embodiments, the plurality of branch layers 130 may be fixedly attached to the central spine 120. In some embodiments, each branch layer of the plurality of branch layers 130 may be fixedly attached to the central spine 120. In some embodiments, the first branch layer 140, the second branch layer 150, and/or the third branch layer 160 may be fixedly attached to the central spine 120. In some embodiments, the first branch layer 140 may be fixedly attached to a first ring member 142 (e.g., FIG. 3) coupled to the central spine 120. In some embodiments, the first ring member 142 may be non-movably coupled to the central spine 120. In some embodiments, the second branch layer 150 may be fixedly attached to a second ring member 152 (e.g., FIG. 3) coupled to the central spine 120. In some embodiments, the second ring member 152 may be non-movably coupled to the central spine 120. In some embodiments, the third branch layer 160 may be fixedly attached to a third ring member 162 (e.g., FIG. 3) coupled to the central spine 120. In some embodiments, the third ring member 162 may be non-movably coupled to the central spine 120. In some alternative configurations, the first branch layer 140, the second branch layer 150, and/or the third branch layer 160 may be monolithically formed with the central spine 120. Other configurations are also contemplated.

In some embodiments, the plurality of branch layers 130 may be directly attached to the central spine 120. In some embodiments, each branch layer of the plurality of branch layers 130 may be directly attached to the central spine 120. In some embodiments, the plurality of branch layers 130 may not be directly attached to each other. In some embodiments, immediately adjacent branch layers of the plurality of branch layers 130 may each be directly attached to the central spine 120, but immediately adjacent branch layers of the plurality of branch layers 130 are not directly attached to each other. In some embodiments, each branch layer of the plurality of branch layers 130 may be considered to be independent of every other branch layer of the plurality of branch layers 130. Other configurations are also contemplated.

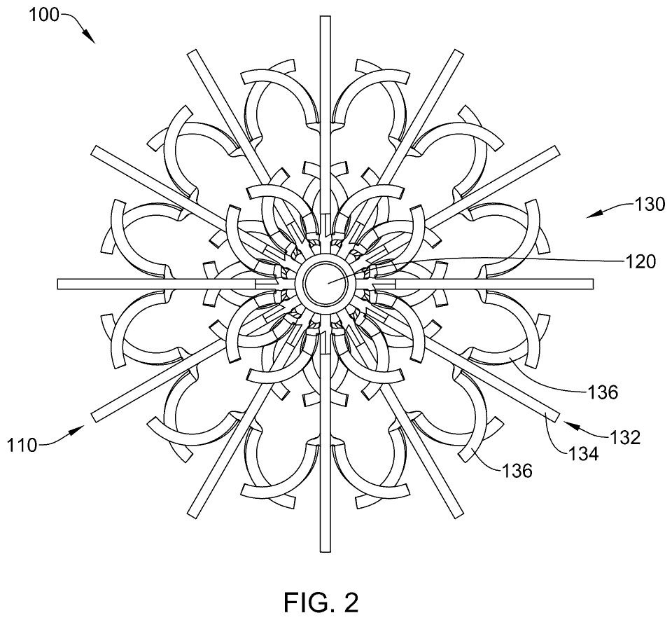

In some embodiments, each branch layer of the plurality of branch layers 130 may comprise a plurality of branches 132 extending radially outward from the central spine 120, as seen in FIGS. 1-2. In some embodiments, each branch of the plurality of branches 132 may comprise a main limb 134 extending radially outward from the central spine 120 and a plurality of side branches 136 extending laterally from the main limb 134. Other configurations are also contemplated.

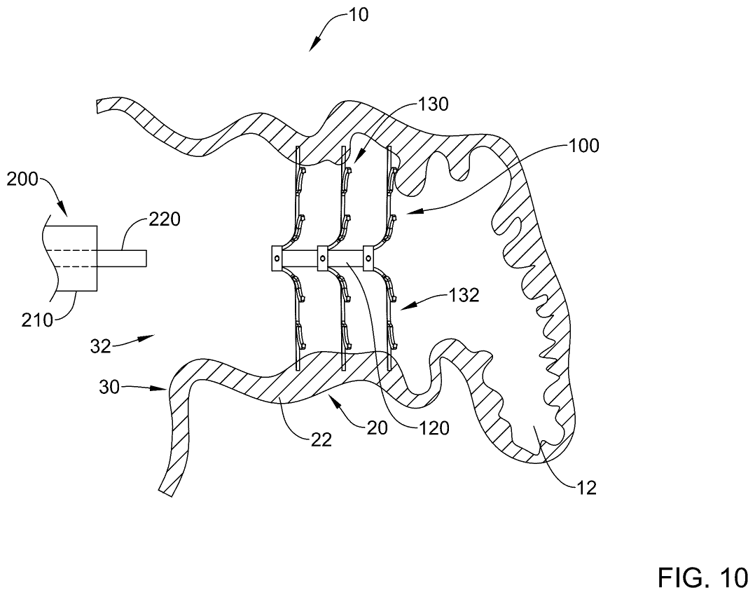

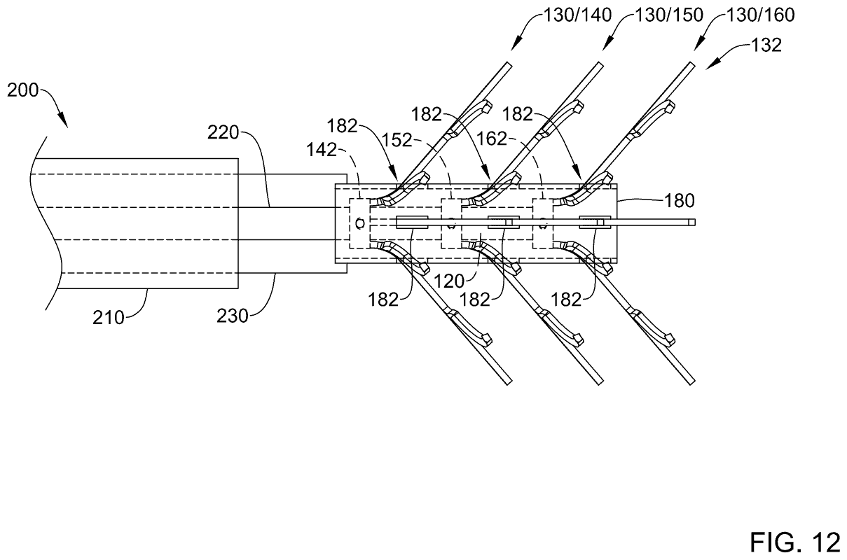

Turning briefly to FIG. 3, in some embodiments, the first branch layer 140 may comprise a first plurality of branches fixedly attached to the first ring member 142. In some embodiments, each branch of the first plurality of branches and/or each main limb thereof may include a base end 144 fixedly attached to the first ring member 142 and a free end 146 disposed opposite the base end 144. Each branch of the first plurality of branches may extend in a distal direction from the base end 144 to the free end 146. In some embodiments, in the collapsed configuration the free end 146 may be disposed in a first radial position relative to a central longitudinal axis of the central spine 120 (e.g., FIG. 8). In some embodiments, in the expanded configuration, the free end 146 may be disposed in a second radial position relative to the central longitudinal axis of the central spine 120 that is radially outward of the first radial position (e.g., FIGS. 10, 12).

In some embodiments, the second branch layer 150 may comprise a second plurality of branches fixedly attached to the second ring member 152. In some embodiments, each branch of the second plurality of branches and/or each main limb thereof may include a base end 154 fixedly attached to the second ring member 152 and a free end 156 disposed opposite the base end 154. Each branch of the second plurality of branches may extend in a distal direction from the base end 154 to the free end 156. In some embodiments, in the collapsed configuration the free end 156 may be disposed in a first radial position relative to a central longitudinal axis of the central spine 120 (e.g., FIG. 8). In some embodiments, in the expanded configuration, the free end 156 may be disposed in a second radial position relative to the central longitudinal axis of the central spine 120 that is radially outward of the first radial position (e.g., FIGS. 10, 12).

In some embodiments, the third branch layer 160 may comprise a third plurality of branches fixedly attached to the third ring member 162. In some embodiments, each branch of the third plurality of branches and/or each main limb thereof may include a base end 164 fixedly attached to the third ring member 162 and a free end 166 disposed opposite the base end 164. Each branch of the third plurality of branches may extend in a distal direction from the base end 164 to the free end 166. In some embodiments, in the collapsed configuration the free end 166 may be disposed in a first radial position relative to a central longitudinal axis of the central spine 120 (e.g., FIG. 8). In some embodiments, in the expanded configuration, the free end 166 may be disposed in a second radial position relative to the central longitudinal axis of the central spine 120 that is radially outward of the first radial position (e.g., FIGS. 10, 12).

In some embodiments, the first ring member 142 may be axially spaced apart from the second ring member 152 and/or the third ring member 162 along the central spine 120. In some embodiments, the second ring member 152 may be axially spaced apart from the first ring member 142 and/or the third ring member 162 along the central spine 120. In some embodiments, the third ring member 162 may be axially spaced apart from the first ring member 142 and/or the second ring member 152 along the central spine 120. In some embodiments, the first ring member 142, the second ring member 152, and the third ring member 162 may be equally spaced apart from each other. In some embodiments, the first ring member 142, the second ring member 152, and the third ring member 162 may be unequally spaced apart from each other. In some embodiments, the first ring member 142, the second ring member 152, and the third ring member 162 may be progressively spaced apart from each other. In some alternative embodiments, the first ring member 142, the second ring member 152, and the third ring member 162 may be in contact with and/or may abut each other. Other configurations are also contemplated.

Returning to FIGS. 1-2, in some embodiments, each branch of the plurality of branches 132 may extend radially outward in a different direction from the central spine 120. In some embodiments, each branch of the plurality of branches 132 may extend radially outward in a different radial direction from the central spine 120. In some embodiments, each branch of the plurality of branches 132 within each branch layer of the plurality of branch layers 130 may extend radially outward in a different direction from the central spine 120. In some embodiments, each branch of the plurality of branches 132 within each branch layer of the plurality of branch layers 130 may extend radially outward in a different radial direction from the central spine 120. In some embodiments, branches within different branch layers of the plurality of branch layers 130 may circumferentially overlap with each other. In some embodiments, branches within each branch layer of the plurality of branch layers 130 may be circumferentially offset from branches within an immediately adjacent branch layer of the plurality of branch layers 130. In some embodiments, branches within each branch layer of the plurality of branch layers 130 may be circumferentially misaligned with and/or offset from branches within every other branch layer of the plurality of branch layers 130. Other configurations are also contemplated.

In some embodiments, the plurality of branches 132 within each branch layer of the plurality of branch layers 130 may be evenly spaced apart radially and/or circumferentially around the central spine 120. In some alternative embodiments, the plurality of branches 132 within each branch layer of the plurality of branch layers 130 may be unevenly spaced apart radially and/or circumferentially around the central spine 120. In some embodiments, the plurality of branches 132 of one or more branch layers of the plurality of branch layers 130 may be evenly spaced apart radially and/or circumferentially around the central spine 120 and one or more branch layers of the plurality of branch layers 130 may be unevenly spaced apart radially and/or circumferentially around the central spine 120. Other configurations are also contemplated.

In some embodiments, the plurality of branch layers 130 may be axially spaced apart from each other in the expanded configuration, as seen in FIG. 3. In some embodiments, the second branch layer 150 may be axially spaced apart from the first branch layer 140 in the expanded configuration. In some embodiments, the third branch layer 160 may be axially spaced apart from the second branch layer 150 in the expanded configuration. In some embodiments, the third branch layer 160 may be axially spaced apart from the first branch layer 140 in the expanded configuration. In some embodiments, the third branch layer 160 may be axially spaced apart from the first branch layer 140 and the second branch layer 150 in the expanded configuration.

In some embodiments, at least one branch layer of the plurality of branch layers 130 may have an outermost radial extent in the expanded configuration that is different from any other branch layers of the plurality of branch layers 130. In some alternative embodiments, different branches and/or main limbs within a branch layer may have different outermost radial extents within that branch layer. Other configurations are also contemplated.

In some preferred embodiments, the expandable framework 110, the central spine 120, the plurality of branch layers 130, the plurality of branches 132, etc. may be formed from nitinol, stainless steel, and the like. Some other suitable but non-limiting materials for the expandable framework 110, the central spine 120, the plurality of branch layers 130, the plurality of branches 132, etc., including but not limited to polymeric materials, metallic materials, shape memory materials, and/or composite materials, are described below.

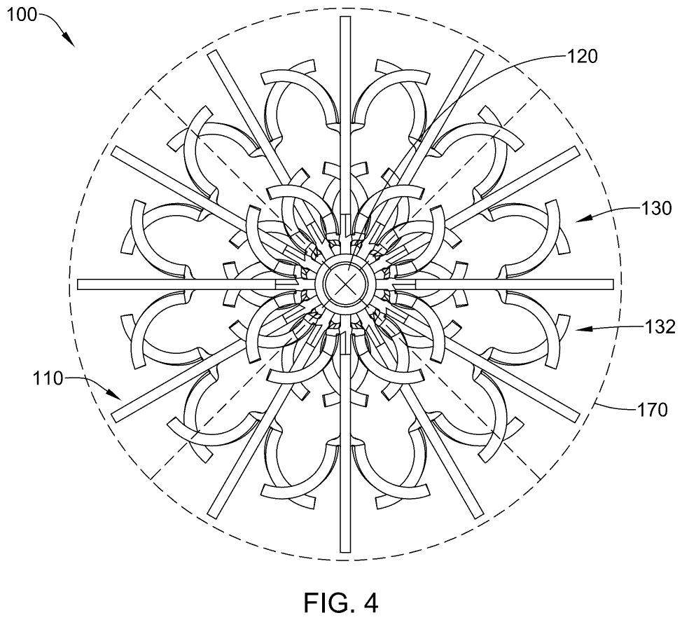

In some embodiments, each branch layer of the plurality of branch layers 130 may comprise at least one layer of foam 170 fixedly attached thereto, as seen in FIGS. 4-7. For clarity, the at least one layer of foam 170 is shown in phantom and all instances of the at least one layer of foam 170 are not shown in each figure. In some embodiments, the first branch layer 140 may comprise at least one layer of foam 170 fixedly attached thereto. In some embodiments, the second branch layer 150 may comprise at least one layer of foam 170 fixedly attached thereto. In some embodiments, the third branch layer 160 may comprise at least one layer of foam 170 fixedly attached thereto. Other configurations are also contemplated.

In some embodiments, each layer of foam of the at least one layer of foam 170 may comprise a single piece of foam surrounding the central spine 120. In some embodiments, each layer of foam of the at least one layer of foam 170 may comprise a single monolithic piece of foam surrounding the central spine 120. In some embodiments, each layer of foam of the at least one layer of foam 170 may form a foam disk when the expandable framework is in the expanded configuration. In some embodiments, each layer of foam of the at least one layer of foam 170 may have an outer perimeter that is circular in shape when the expandable framework is in the expanded configuration. Other configurations and/or shapes are also contemplated.

In some embodiments, each layer of foam of the at least one layer of foam 170 may comprise a plurality of individual pieces of foam disposed circumferentially around the central spine 120. In some embodiments, each branch of the plurality of branches 132 within each branch layer of the plurality of branch layers 130 may comprise and/or may have a single piece of foam (e.g., only one piece of the plurality of individual pieces of foam) fixedly attached thereto.

In some embodiments, the plurality of individual pieces of foam within each layer of foam may engage with each other when the expandable framework is in the expanded configuration to form a single complete layer of foam surrounding the central spine 120. In some embodiments, the plurality of individual pieces of foam within each layer of foam may be circumferentially spaced apart from each other when the expandable framework is in the expanded configuration to form a discontinuous layer of foam surrounding the central spine 120. In some embodiments, the plurality of individual pieces of foam within each layer of foam may each have a shape that is triangular. In some embodiments, the plurality of individual pieces of foam within each layer of foam may each have a shape that is trapezoidal. In some embodiments, the plurality of individual pieces of foam within each layer of foam may each have a shape that is wedge-shaped and/or pie-shaped. Other configurations and/or shapes are also contemplated.

The at least one layer of foam 170 may be configured to shift from a first configuration (e.g., a compressed configuration) to a second configuration (e.g., an expanded configuration). In some embodiments, the at least one layer of foam 170 may be constrained in the first configuration (e.g., the compressed configuration) by a delivery device. In some embodiments, the at least one layer of foam 170 may be configured to shift from the first configuration (e.g., the compressed configuration) to the second configuration (e.g., the expanded configuration) when unconstrained.

In some embodiments, the at least one layer of foam 170 may be configured to shift from the first configuration (e.g., the compressed configuration) to the second configuration (e.g., the expanded configuration) in vivo. In some embodiments, the at least one layer of foam 170 may be configured to adapt and conform to the left atrial appendage 10 (e.g., FIG. 8) and/or surrounding anatomy when shifting from the first configuration (e.g., the collapsed configuration) toward and/or to the second configuration (e.g., the expanded configuration) in vivo, as seen in FIGS. 10 and 12.

In some embodiments, the at least one layer of foam 170 may be configured to shift from the first configuration (e.g., the compressed configuration) to the second configuration (e.g., the expanded configuration) when exposed to a stimulus, such as a preselected temperature, a fluid, or a combination thereof. In some embodiments, the at least one layer of foam 170 may be formed from a shape memory material. In some embodiments, each piece of foam of the at least one layer of foam 170 may be formed from a different shape memory material. Other configurations are also contemplated.

In some embodiments, the at least one layer of foam 170 may have a first overall volume in the first configuration (e.g., the compressed configuration). In some embodiments, the at least one layer of foam 170 may have a second overall volume in the second configuration (e.g., the expanded configuration) different from the first overall volume. In some embodiments, the second overall volume may be about 10% different, about 20% different, about 30% different, about 40% different, about 50% different, about 75% different, about 100% different, about 125% different, about 150% different, about 200% different, etc. from the first overall volume. In at least some embodiments, the second overall volume may be greater than the first overall volume.

In some embodiments, the at least one layer of foam 170 may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed or delivery size. In some examples, the collapsibility ratio of the shape memory polymer and/or the shape memory foam may be at least 5 times, at least 7 times, at least 8 times, at least 9 times, at least 10 times, at least 12 times, or more. In one non-limiting example, a piece of expandable foam may have an outer diameter of about 32 millimeters (1.260 inches) in the expanded configuration and about 3 millimeters (0.118 inches) in the compressed configuration, producing a collapsibility ratio of at least 10 times (e.g., at least 10:1). Other configurations are also contemplated. In at least some embodiments, the at least one layer of foam 170 may be configured as open celled foam.

In some embodiments, the at least one layer of foam 170 and/or the shape memory foam may be formed from a biocompatible material. In at least some embodiments, the at least one layer of foam 170 may be non-biodegradable and/or non-bioabsorbable. In some alternative embodiments, the at least one layer of foam 170 may be biodegradable and/or bioabsorbable over time. In some embodiments, the at least one layer of foam 170 may be configured to promote endothelization and/or tissue ingrowth. In some embodiments, the at least one layer of foam 170 may include a coating, a material, and/or a component that promotes endothelization and/or tissue ingrowth. Other configurations are also contemplated.

In some embodiments, the at least one layer of foam 170 may be configured to prevent thrombus formation (e.g., within the left atrial appendage 10 and/or on each layer of foam of the at least one layer of foam 170). In some embodiments, the at least one layer of foam 170 may include an anti-thrombus agent(s) and/or medicament(s). In some embodiments, the at least one layer of foam 170 may be configured to absorb blood and/or bodily fluid(s). In some embodiments, the at least one layer of foam 170 may be configured to trap thrombus. In some embodiments, the at least one layer of foam 170 may be configured to promote tissue ingrowth and/or endothelization. Other configurations are also contemplated.

In some embodiments, the at least one layer of foam 170 may be unconstrained by outside forces and/or structure(s) in the first configuration (e.g., the collapsed configuration). In some embodiments, the at least one layer of foam 170 may be self-maintained in the first configuration (e.g., the collapsed configuration) by shape memory properties. Other configurations are also contemplated.

In some embodiments, the at least one layer of foam 170 may optionally comprise a radiopaque marker coupled thereto and/or embedded therein. In some embodiments, the radiopaque marker may comprise a radiopaque substance or a radiopaque material disposed within at least one layer of foam 170 (e.g., the at least one layer of foam 170 may be doped with and/or may include the radiopaque substance or the radiopaque material).

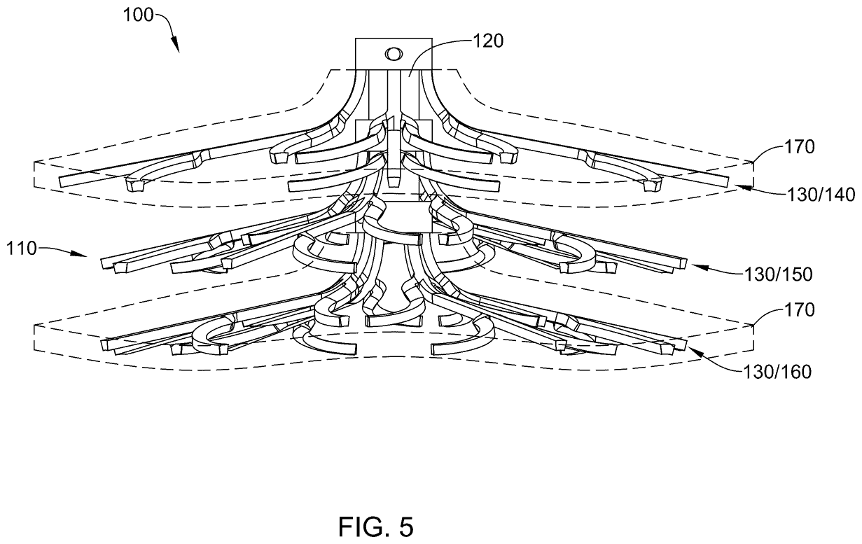

In some embodiments, the at least one layer of foam 170 may be fixedly attached to a top surface of its respective branch layer, as seen in FIG. 5. In some embodiments, the at least one layer of foam 170 fixedly attached to the first branch layer 140 may be fixedly attached to a top surface of the first branch layer 140 and/or the first plurality of branches thereof. In some embodiments, the at least one layer of foam 170 fixedly attached to the second branch layer 150 may be fixedly attached to a top surface of the second branch layer 150 and/or the second plurality of branches thereof. In some embodiments, the at least one layer of foam 170 fixedly attached to the third branch layer 160 may be fixedly attached to a top surface of the third branch layer 160 and/or the third plurality of branches thereof. Other configurations are also contemplated.

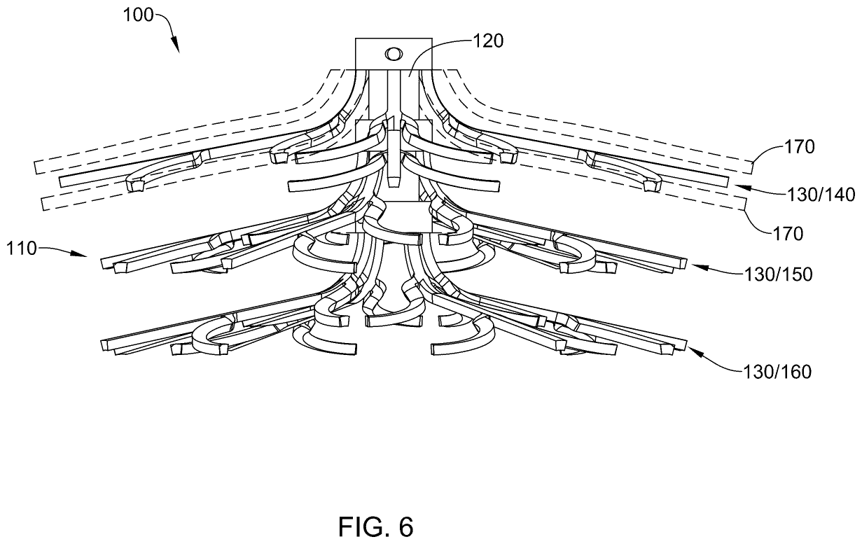

In some embodiments, the at least one layer of foam 170 may be fixedly attached to a bottom surface of its respective branch layer, as seen in FIG. 6. In some embodiments, the at least one layer of foam 170 fixedly attached to the first branch layer 140 may be fixedly attached to a bottom surface of the first branch layer 140 and/or the first plurality of branches thereof. In some embodiments, the at least one layer of foam 170 fixedly attached to the second branch layer 150 may be fixedly attached to a bottom surface of the second branch layer 150 and/or the second plurality of branches thereof. In some embodiments, the at least one layer of foam 170 fixedly attached to the third branch layer 160 may be fixedly attached to a bottom surface of the third branch layer 160 and/or the third plurality of branches thereof. Other configurations are also contemplated.

In some alternative embodiments, the at least one layer of foam 170 may comprise two layers of foam, wherein a first layer of foam is fixedly attached to a top surface and a second layer of foam is fixedly attached to a bottom surface of their respective branch layer such that the first layer of foam is spaced apart from the second layer of foam. In some embodiments, the at least one layer of foam 170 fixedly attached to the first branch layer 140 may comprise two layers of foam, wherein a first layer of foam is fixedly attached to a top surface of the first branch layer 140 and a second layer of foam is fixedly attached to a bottom surface of the first branch layer 140 such that the first layer of foam is spaced apart from the second layer of foam. In some embodiments, the at least one layer of foam 170 fixedly attached to the second branch layer 150 may comprise two layers of foam, wherein a first layer of foam is fixedly attached to a top surface of the second branch layer 150 and a second layer of foam is fixedly attached to a bottom surface of the second branch layer 150 such that the first layer of foam is spaced apart from the second layer of foam. In some embodiments, the at least one layer of foam 170 fixedly attached to the third branch layer 160 may comprise two layers of foam, wherein a first layer of foam is fixedly attached to a top surface of the third branch layer 160 and a second layer of foam is fixedly attached to a bottom surface of the third branch layer 160 such that the first layer of foam is spaced apart from the second layer of foam. Other configurations are also contemplated.

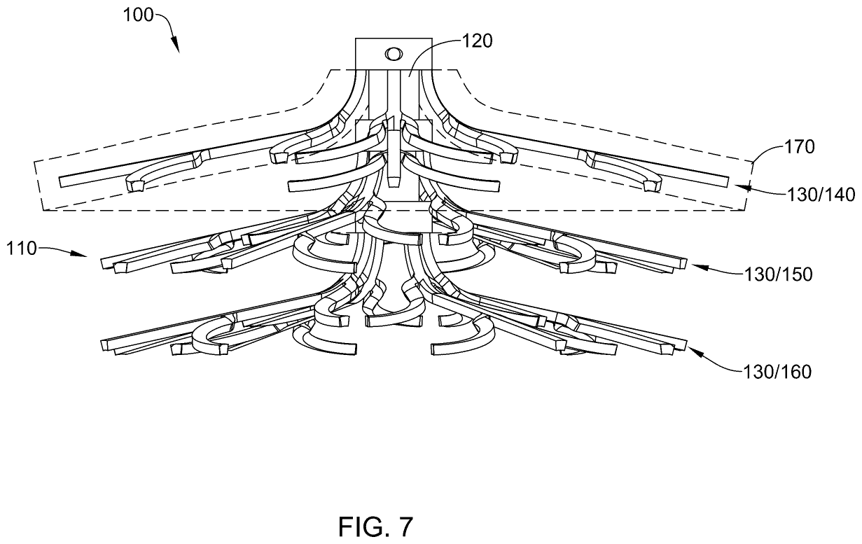

In some embodiments, each branch layer of the plurality of branch layers 130 may be embedded within its respective at least one layer of foam 170, as seen in FIG. 7. In at least some embodiments, each branch layer of the plurality of branch layers 130 may be embedded within a single layer of foam (e.g., within only one layer of foam). In some embodiments, the first branch layer 140 may be embedded within its respective at least one layer of foam 170. In some embodiments, the first branch layer 140 may be sandwiched between two layers of foam (e.g., the first layer of foam fixedly attached to the top surface of the first branch layer 140 and the second layer of foam fixedly attached to the bottom surface of the first branch layer 140) such that the two layers of foam are in intimate contact with each other. In some embodiments, the second branch layer 150 may be embedded within its respective at least one layer of foam 170. In some embodiments, the second branch layer 150 may be sandwiched between two layers of foam (e.g., the first layer of foam fixedly attached to the top surface of the second branch layer 150 and the second layer of foam fixedly attached to the bottom surface of the second branch layer 150) such that the two layers of foam are in intimate contact with each other. In some embodiments, the third branch layer 160 may be embedded within its respective at least one layer of foam 170. In some embodiments, the third branch layer 160 may be sandwiched between two layers of foam (e.g., the first layer of foam fixedly attached to the top surface of the third branch layer 160 and the second layer of foam fixedly attached to the bottom surface of the third branch layer 160) such that the two layers of foam are in intimate contact with each other. Other configurations are also contemplated.

FIG. 8 schematically illustrates selected aspects of a left atrial appendage 10. It shall be understood that the left atrial appendage 10 of FIG. 8 is merely exemplary, and other geometries, shapes, sizes, etc. for the left atrial appendage 10 are common and may vary from patient to patient. The left atrial appendage 10 may be formed as a small pouch or extension attached to and extending from the left atrium of a patient's heart.

The left atrial appendage 10 may include a longitudinal axis arranged along a depth of a main body 20 of the left atrial appendage 10. The main body 20 may include a side wall 22 and an ostium 30 forming a proximal mouth 32. In some embodiments, a lateral extent of the main body 20 and/or the ostium 30 may be smaller or less than a depth of the main body 20 along the longitudinal axis, or a depth of the main body 20 may be greater than a lateral extent of the main body 20 and/or the ostium 30. In some embodiments, the left atrial appendage 10 may narrow quickly along the depth of the main body 20 or the left atrial appendage 10 may maintain a generally constant lateral extent along a majority of the depth of the main body 20.

In some embodiments, the left atrial appendage 10 may include a distalmost region 12 formed or arranged as a tail-like element associated with a distal portion of the main body 20. In some embodiments, the distalmost region 12 may protrude radially or laterally away from the longitudinal axis and/or the main body 20.

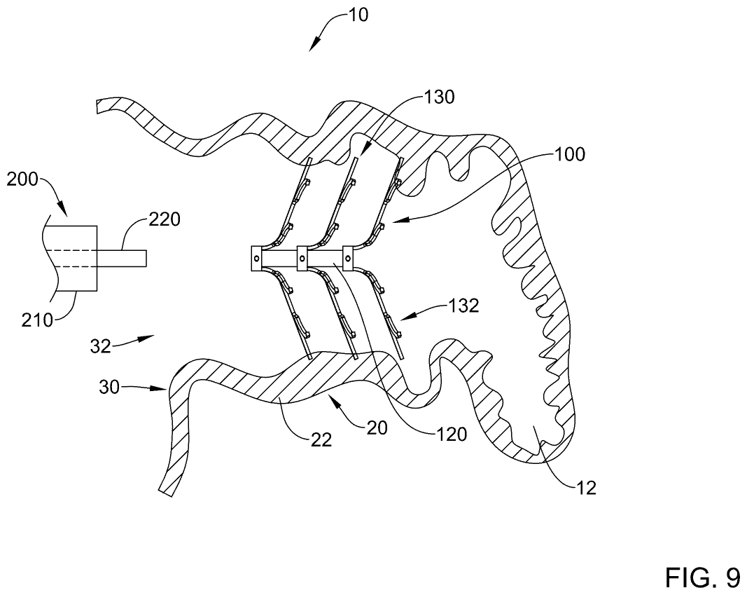

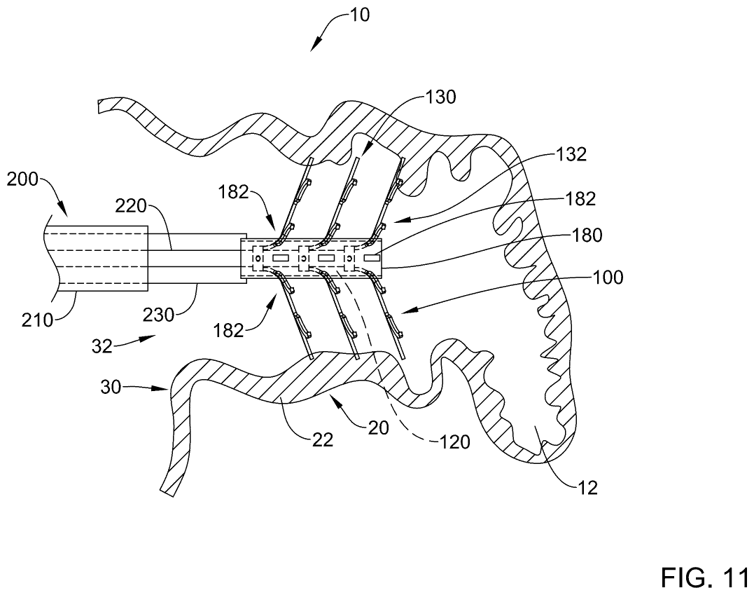

FIG. 8 also illustrates a delivery device 200 comprising a tubular sheath 210 having a lumen extending therethrough. The implant 100 is disposed within the lumen of the tubular sheath 210 in a delivery configuration (e.g., the collapsed configuration of the expandable framework 110). For clarity, the at least one layer of foam 170 is not shown in FIGS. 8-15 but shall be understood to take any of the forms disclosed herein. The implant 100 may be advanced into the left atrial appendage 10 using the delivery device 200. Thereafter, the implant 100 may be deployed within the left atrial appendage 10, as seen in FIGS. 9 and 11. In some embodiments, the delivery device 200 may comprise a pusher 220 slidably disposed within the tubular sheath 210. In some embodiments, the implant 100 may be deployed by translating the pusher 220 distally within and/or relative to the tubular sheath 210 to push the implant 100 out a distal end of the tubular sheath 210. In some embodiments, the pusher 220 may be held in place while the tubular sheath 210 is withdrawn proximally over and/or relative to the pusher 220 to deploy the implant 100 out the distal end of the tubular sheath 210. Other configurations are also contemplated.

In some embodiments, the expandable framework 110 may be self-biased toward and/or to the expanded configuration, as seen in FIGS. 9-10. In some embodiments, the expandable framework 110 may be formed from a shape memory material. In some embodiments, the expandable framework 110 may be configured to pull the central spine 120 deeper into the main body 20 of the left atrial appendage 10 as it shifts toward the expanded configuration.

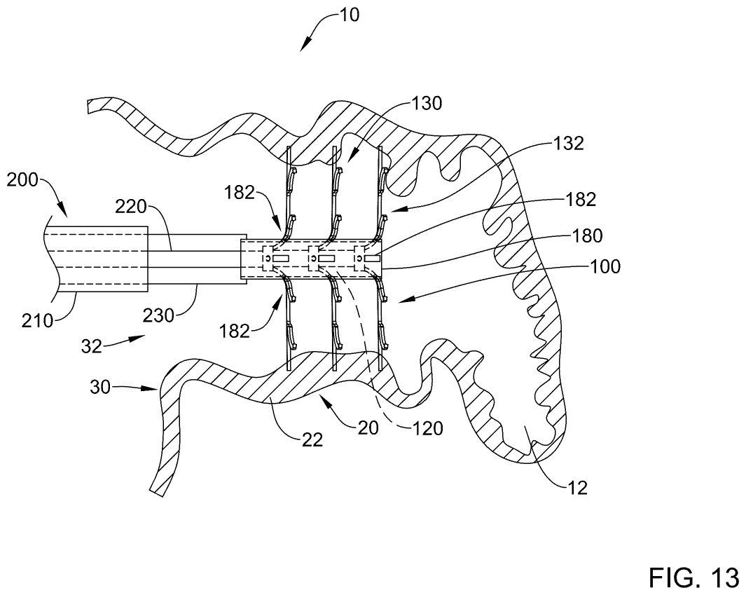

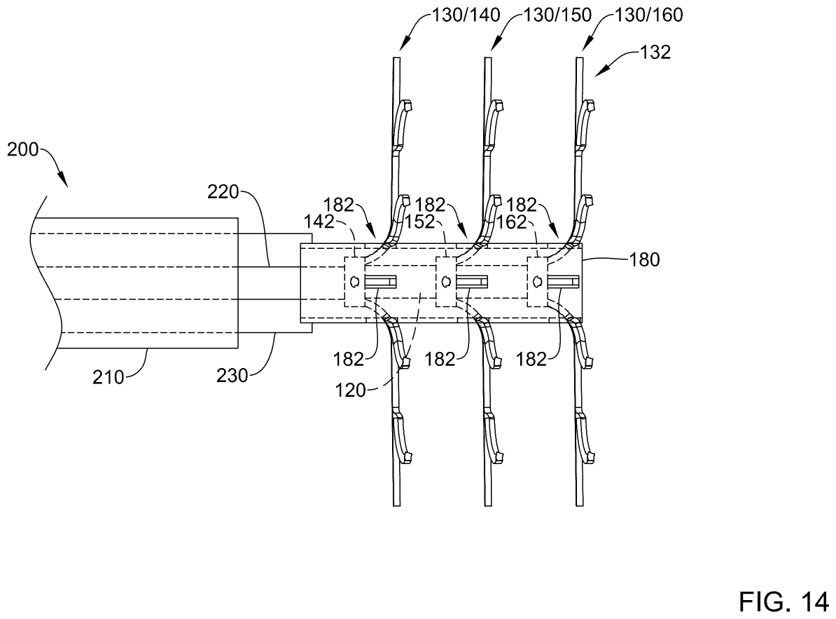

In some embodiments, the expandable framework 110 may be manually actuatable between the collapsed configuration and the expanded configuration, as seen in FIGS. 11-14. It should be noted that some branches of the plurality of branches 132 have been omitted from FIGS. 11 and 13, and some structure of the plurality of branches 132 has been omitted from FIGS. 12 and 14, in the interest of clarity. In some embodiments, the implant 100 and/or the expandable framework 110 may comprise a tubular member 180 disposed radially outward of the central spine 120. In some embodiments, longitudinal slots 182 formed in the tubular member 180 may surround each branch of the plurality of branches 132 (e.g., each branch may extend through one of the longitudinal slots 182), as seen in FIGS. 12 and 14.

In some embodiments, longitudinal slots 182 formed in the tubular member 180 may surround each branch of the first plurality of branches of the first branch layer 140. In some embodiments, longitudinal slots 182 formed in the tubular member 180 may surround each branch of the second plurality of branches of the second branch layer 150. In some embodiments, longitudinal slots 182 formed in the tubular member 180 may surround each branch of the third plurality of branches of the third branch layer 160.

In some embodiments, each branch of the plurality of branches 132 may be disposed at a proximal end of its respective longitudinal slot (e.g., ref. 182) when the expandable framework is in the collapsed configuration, as seen in FIG. 12. The central spine 120 may be axially movable relative to the tubular member 180 to shift the expandable framework 110 between the collapsed configuration (e.g., FIGS. 11-12) and the expanded configuration (e.g., FIGS. 13-14). As seen in FIG. 14, in the expanded configuration, each branch of the plurality of branches 132 may be disposed at a distal end of its respective longitudinal slot (e.g., ref. 182). As each branch of the plurality of branches 132 is urged, pushed, and/or biased against the distal end of its respective longitudinal slot, the plurality of branches 132 of each branch layer may be deflected and/or shifted radially outward toward the expanded configuration.

In some embodiments, the delivery device 200 may comprise a holding element 230. In some embodiments, the holding element 230 may be slidably disposed within the lumen of the tubular sheath 210. In some embodiments, the holding element 230 may be configured to releasably hold, grasp, and/or secure the tubular member 180 relative to the tubular sheath 210 and/or the pusher 220. In some embodiments, the holding element 230 may be configured to releasably hold the tubular member 180 in a fixed position while the pusher 220 is translated and/or advanced distally to actuate the expandable framework 110 from the collapsed configuration toward and/or to the expanded configuration. Thereafter, the holding element 230 may be disengaged from the tubular member 180 to completely release the implant 100 within the left atrial appendage 10. In some embodiments, the holding element 230 may be a tubular structure configured to engage with (e.g., snap onto, frictionally engage, etc.) the tubular member 180. In some embodiments, the holding element 230 may comprise a plurality of arms having distal tips configured to engage with (e.g., snap onto, extend into a wall of, extend through a wall of, etc.) the tubular member 180. Other configurations are also contemplated.

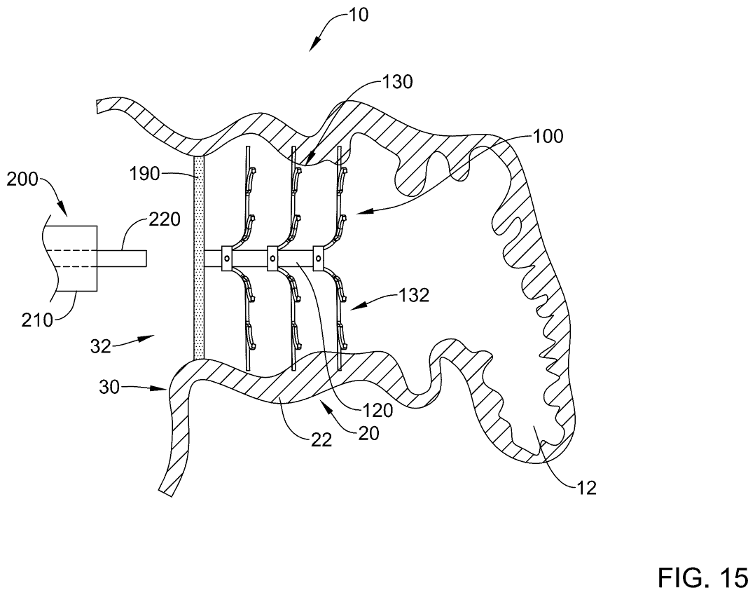

In some embodiments, the implant 100 may comprise a continuous foam disk 190 disposed at a proximal end of the implant 100, as seen in FIG. 15. For the purpose of this disclosure, a “continuous foam disk” may shall be understood to comprise a single monolithic piece of foam and shall exclude foam disks or layers of foam comprising multiple discrete pieces of foam. The single monolithic piece of foam may be open-celled foam or close-celled foam in accordance with the disclosure. In some embodiments, the continuous foam disk 190 may be disposed proximal of the plurality of branch layers 130. In some embodiments, the continuous foam disk 190 may be fixedly attached directly to the expandable framework 110, the central spine 120, the first ring member 142, and/or the tubular member 180 (not shown in FIG. 15), as desired. In some embodiments, the continuous foam disk 190 may be configured to shift from a radially collapsed configuration to a radially expanded configuration (e.g., FIG. 15). In the radially expanded configuration, the continuous foam disk 190 may engage with, contact, and/or abut the side wall 22 of the left atrial appendage 10. In some embodiments, the continuous foam disk 190 may be configured to form a “cap” at and/or adjacent to the ostium 30 of the left atrial appendage 10, thereby closing off the left atrial appendage 10 from the left atrium.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the implant, the expandable framework, the central spine, the plurality of branch layers, the plurality of branches, the tubular member, the delivery device, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

The foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. The foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D, L-lactide, poly(beta-hydroxybutyrate), polygluconate including poly-D-gluconate, poly-L-gluconate, poly-D, L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerization of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine-and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. An implant for occluding a left atrial appendage, comprising:
an expandable framework configured to shift from a collapsed configuration to an expanded configuration, wherein the expandable framework comprises a central spine and a plurality of branch layers extending radially outward from the central spine;
wherein each branch layer of the plurality of branch layers comprises at least one layer of foam fixedly attached thereto.

2. The implant of claim 1, wherein each branch layer of the plurality of branch layers comprises a plurality of branches extending radially outward from the central spine.

3. The implant of claim 2, wherein each branch of the plurality of branches within each branch layer extends radially outward in a different direction from the central spine.

4. The implant of claim 1, wherein the at least one layer of foam is fixedly attached to a top surface of its respective branch layer.

5. The implant of claim 1, wherein the at least one layer of foam is fixedly attached to a bottom surface of its respective branch layer.

6. The implant of claim 1, wherein each branch layer is embedded within its respective at least one layer of foam.

7. The implant of claim 1, wherein each layer of foam of the at least one layer of foam comprises a plurality of individual pieces of foam disposed circumferentially around the central spine.

8. The implant of claim 7, wherein the plurality of individual pieces of foam within each layer of foam engage with each other when the expandable framework is in the expanded configuration to form a single complete layer of foam surrounding the central spine.

9. The implant of claim 1, wherein each layer of foam of the at least one layer of foam comprises a single piece of foam surrounding the central spine.

10. The implant of claim 1, wherein branches within each branch layer of the plurality of branch layers are circumferentially offset from branches of an adjacent branch layer of the plurality of branch layers.

11. An implant for occluding a left atrial appendage, comprising:
an expandable framework configured to shift from a collapsed configuration to an expanded configuration, wherein the expandable framework comprises a central spine and a plurality of branch layers extending radially outward from the central spine;
wherein each branch layer of the plurality of branch layers comprises at least one layer of foam fixedly attached thereto;
wherein branches within each branch layer are circumferentially misaligned with branches within every other branch layer.

12. The implant of claim 11, wherein the plurality of branch layers is axially spaced apart from each other in the expanded configuration.

13. The implant of claim 11, wherein at least one branch layer of the plurality of branch layers has an outermost radial extent in the expanded configuration that is different from any other branch layers of the plurality of branch layers.

14. The implant of claim 11, wherein each branch layer comprises a plurality of branches, and each branch of the plurality of branches comprises a main limb extending radially outward from the central spine and a plurality of side branches extending laterally from the main limb.

15. An implant for occluding a left atrial appendage, comprising:
an expandable framework configured to shift from a collapsed configuration to an expanded configuration, wherein the expandable framework comprises a central spine and a plurality of branch layers extending radially outward from the central spine;
wherein the plurality of branch layers comprises a first branch layer, a second branch layer axially spaced apart from the first branch layer in the expanded configuration, and third branch layer axially spaced apart from the first branch layer and the second branch layer in the expanded configuration;
wherein the first branch layer comprises at least one layer of foam fixedly attached thereto, the second branch layer comprises at least one layer of foam fixedly attached thereto, and the third branch layer comprises at least one layer of foam fixedly attached thereto;
wherein the first branch layer comprises a first plurality of branches fixedly attached to a first ring member coupled to the central spine, the second branch layer comprises a second plurality of branches fixedly attached to a second ring member coupled to the central spine, and the third branch layer comprises a third plurality of branches fixedly attached to a third ring member coupled to the central spine.

16. The implant of claim 15, wherein each branch of the first plurality of branches includes a base end fixedly attached to the first ring member and a free end disposed opposite the base end;
wherein each branch of the first plurality of branches extends in a distal direction from the base end to the free end;
wherein in the collapsed configuration the free end is disposed at a first radial position relative to a central longitudinal axis of the central spine, and wherein in the expanded configuration the free end is disposed at a second radial position relative to the central longitudinal axis of the central spine that is radially outward of the first radial position.

17. The implant of claim 15, wherein the first ring member is axially spaced apart from the second ring member and the third ring member.

18. The implant of claim 15, wherein the expandable framework is self-biased toward the expanded configuration.

19. The implant of claim 15, wherein the expandable framework is manually actuatable between the collapsed configuration and the expanded configuration.

20. The implant of claim 19, wherein the expandable framework comprises a tubular member disposed radially outward of the central spine;
wherein longitudinal slots formed in the tubular member surround each branch of the first plurality of branches, each branch of the second plurality of branches, and each branch of the third plurality of branches;
wherein the central spine is axially movable relative to the tubular member to shift the expandable framework between the collapsed configuration and the expanded configuration.
